(19) United States
(12) Patent Application Publication
(10) Pub. No.: US 2012/0239000 A1
(43) Pub. Date: Sep. 20, 2012

(54) Drug Delivery Method Via Brain Extracellular Space and a Device Thereof

(76) Inventor: Hongbin Han, Beijing (CN)

(21) Appl. No.: 13/511,673

(22) PCT Filed: Dec. 7, 2009
(86) PCT No.: PCT/CN2009/075350

§ 371 Date: May 24, 2012

(51) Int. Cl. A61M 5/168; A61B 5/055

(52) U.S. Cl. 604/506; 604/500; 604/65; 604/67

(57) ABSTRACT

The present invention relates to a method for drug delivery via brain extracellular space (ECS). It includes putting head of patient in an imaging apparatus; generating dynamic images of the brain via the imaging apparatus; delivering drugs into brain ECS of the patient according to the dynamic images and the drugs get to the therapeutic target by simple diffusion along concentration gradient. The present invention also relates to a device for drug delivery via brain ECS. Basing on the self-diffusion delivery (SDD) of drugs in brain ECS, the present invention can deliver drug into the ECS of the brain's relatively safe area in low speed, with small amounts of doses and without extra stress. Therefore, the present invention can reduce delivery time and dose of drugs, relieve injection pressure, decrease damages on normal brain tissue, and reduce the cost of treatment observably.

### FIELD OF THE INVENTION

The present invention relates to a drug delivery method via brain extracellular space. In particular, it related to a method using self-diffusion delivery (SDD) of drug in brain extracellular space to make the drug to reach the target brain tissue and produce effect. The present invention also relates to a drug delivery device via brain extracellular space.

### BACKGROUND OF THE INVENTION

In the therapeutic process of acute ischemic stroke, the effectiveness of drug delivery is very important. Since the 1950s, scientists have studied the cytidine diphosphate as neuroprotective drugs. The Takeda company from Japan used Nicholin (Citicoline) to cure the disturbances of consciousness successfully. In 2002, Davalos etc. analysed the effect of the peroral citicoline for curing ischemic stroke by evidence based medicine. In 2005, Hurtado etc. made the model of focal brain ischemia using adult male Fischer rat, then injected CDPC (Cytidine-5′-diphosphate choline) into the abdominal of the Fischer rat and blocked the middle cerebral artery one hour after the injection. The Hurtado's results demonstrate that injecting CDPC with different doses (e.g. 0.5 g/kg, 1 g/kg and 2 g/kg) can reduce infarct size of the neostriatum. According the study of Hurtado etc. in 2008, comparing with the control group, 48 hours-brain infarct size can be reduced significantly if CDPC are injected into the abdominal cavity of the Fischer rats with the dose of 2 g/kg four hours after embolism by focal brain ischemia. All of the preceding clinic research and animal experiment confirmed that CDPC has a neuroprotection function. Mechanism research shows that CDPC plays the role of protecting brain from ischemia and treat injury mainly through stabilizing the cell membrane, suppressing the release of free fatty acid, reducing free radical generation and inhibiting cell apoptosis.

Because of the blood-brain-barrier (BBB), macromolecules and polar molecules are difficult to diffuse from blood to brain tissue during the treatment of brain parenchyma disease. Therefore, the absorption and the utilization ratio of drug delivered via vessel are limited and the healing effect is unsatisfactory. For example, the mechanism of neuron injury and apoptosis under ischemia are confirmed very clearly both in the molecular and cellular level and many countries spending a large amount of money to develop various neuroprotective drugs in allusion to different physiological process of brain ischemic injury, but the result was not ideal.

The CDPC is a micromolecule and only has 510.31 dalton molecular weight, and it's difficult for CDPC to cross the BBB because of its strong polarity. Studies have shown that the intake of CDPC delivered via oral and intravenous injection is very poor. As shown in animal experiments, the intake rate of CDPC delivered via oral method is only 0.5% and the intake rate of CDPC delivered via intravenously methods is 2%, which is still very low. Because the neuroprotection of citicoline is dose-dependent, the intake of CDPC absorbed by brain tissue is one of the key factors of the efficacy.

Except for the blocking effect of BBB, it is also difficult for drug to transport to the infarct region because of the low volume ratio of the cerebral microvascular in total brain, only 3%, and the hypoperfusion before recanalization. The blocking effect of BBB will also reduce the effective concentration of citicoline even if the drugs reach the infarction regions.

The intake of citicoline is improved usually by increasing the dosage of drugs and enhancing the permeability of blood brain barrier via liposome. These methods can make the intake rate of drugs increased to 23%. Since the citicoline can be decomposed and used to synthesize the excitability neurotransmitter-acetylcholine, heavy doses of citicoline may cause excessive acetylcholine generation and result in the super stimulation of nerve muscle. In addition, adding liposomes or other external drugs to open the BBB selectively and increase the permeability thereof, may increase the possibility of the side effects and treatment complications. So far, the BBB makes most of the neuroprotective agents fail.

U.S. patent application Ser. No. 5,720,720 provides a method of convection-enhanced delivery of drug (CED) in order to avoid the obstruction of the BBB. In CED, the brain tissue is supposed to be a solid medium, while drugs in solid state media can only move to target area via the driving of pressure. CED is a drug delivery method in which drug is directly inject into the brain, it uses external pressure to inject the drug. The drug is injected with a certain speed from the injection point for a few minutes or even hours into the brain tissue and causes a pressure gradient between the injection point and the target area, so the drug can arrive at the target area along the gradient pressure.

Although the CED method can avoid the restrictions of the BBB and improve the utilization rate of the drug, the delivery method needs to deliver drugs continuously under pressure, and the requirements about the position of drug delivery (puncture points), the total amount and speed of delivered drug are very strict. The CED is applicable for macromolecule drugs having a generally the puncture position far from the target area, so in order to keep the drug concentration in the target region, we need to inject a large amount of drugs. For example, if we inject the drugs with a speed of 0.5-15 μL/min continuously, the total volume of the drug injected will be up to 600 μL.

The CED method has obtained effects in treatment of tumor, however, in recent years, medical study shows that the brain is not all a solid medium, and a long term and heavy dose of drugs may cause brain injury, such as brain interstitial edema, brain volume increases or cerebral hernia. In addition, the problem of drugs reflux due to the large pressure and the high speed of the drug delivery must be solved using the CED method. At the same time, the heavy doses, long term and continuity of drug delivery have greatly increased the curative cost. All of the above disadvantages greatly limit the spreading of CED method in clinic.

### SUMMARY OF THE INVENTION

The technical problem to be solved by the present invention is to provide a drug delivery method for CNS diseases via brain extracellular space (ECS). The method can deliver the drug to the brain ECS in the brain's relatively safe areas in low speed, with small doses and without extra stress via the self-diffusion delivery (SDD) of the drug in brain ECS. Therefore, the method can reduce delivery time, drug dose and injection pressure, thus decrease the damage to a normal brain tissue and reduce the cost of treatment observably.

Another technical problem to be solved by the present invention is to provide a drug delivery device via brain ECS. The device can determine the accurate position of the drug infusion in brain region, and real-time monitor the process of drug delivery using the MRI images of brain ECS. Therefore, the device can realize the accurate, timely and effective treatment for brain disease of the patient.

The present invention provides a drug delivery method for via brain extracellular space including: putting brain of a patient in an imaging apparatus; generating dynamic images of the brain with the imaging apparatus; delivering the drug to the brain extracellular space of the patient according to the dynamic image and the drug moving to the therapeutic target of the patient via self-diffusion along the concentration gradient.

Certain exemplary embodiments can provide a method, in which the drug is delivered to the brain extracellular space via brain puncturing.

Certain exemplary embodiments can provide a method, in which the drug is CDPC.

Certain exemplary embodiments can provide a method, in which the imaging apparatus is magnetic resonance imager.

The present invention further provides a drug delivery device via brain extracellular space. The device comprises: an imaging apparatus configured to get dynamic images of the patient; a drug delivery apparatus configured to deliver the drug to the brain extracellular space of the patient and a controlling apparatus which is connected with the imaging apparatus and the drug delivery apparatus. The controlling apparatus includes: a control unit configured to receive imaging signals from the imaging apparatus and control the speed and the total dose of the drug via connecting with the drug delivery apparatus; and a monitor unit which is connected with the control unit and configured to display the images from the imaging apparatus.

Certain exemplary embodiments can provide a device, in which the drug delivery apparatus comprises: a puncture needle configured to import the drugs to the brain extracellular space of the patient; and a dosing pump, which is connected with the puncture needle and configured to deliver the drugs to the puncture needle.

Certain exemplary embodiments can provide a device, in which the device further includes a stereotaxic apparatus to fix the position of the patient head in the imaging apparatus and the puncture needle can be provided in the stereotaxic apparatus.

Certain exemplary embodiments can provide a device, in which the stereotaxic apparatus is made of brass.

Certain exemplary embodiments can provide a device, in which the controlling apparatus further comprises an input unit which is connected to the control unit and configured to input controlling signal to the imaging apparatus and the drug delivery apparatus.

The present invention deliveries the drug via the extracellular space. It uses the extracellular space and the interstitial fluid as the diffusion media for the drug. The drug which has a relatively small molecular weight can reach the target by self-diffusion without external pressure, and thus have curative effects.

Compared with the CED or other traditional methods, the present method only need a small amount of drug to achieve treatment effect, therefore reduces the damage to the target tissue and transporting tissue as well as decreases curative costs.

Since the present device dynamicly monitor the patient's brain, the location of the drug to be administrated, the dosage thereof, the treatment processes and the treat result can be monitored, therefore realizes a safe prevention and treatment for brain diseases such as cerebral infarction.

### BRIEF DESCRIPTION OF THE DRAWINGS

FIG. 1(a) shows the brain slice undergoing TTC staining of the sham group 12 hours after surgery.

FIG. 1(b) shows the brain slice undergoing TTC staining of the control group 12 hours after the surgery.

FIG. 1(c) shows the brain slice undergoing TTC staining of the i.p. group 12 hours after the surgery.

FIG. 1(d) shows the brain slice undergoing TTC staining of the i.c. group 12 hours after the surgery.

FIG. 2 shows the ratio of the infarct volume of the control group, i.p group and the i.c group 12 hours after cerebral ischemia.

FIG. 3 schematically illustrates a device for drug delivery via brain extracellular space.

FIG. 4 shows the MRI image of the rat's brain being punctured.

FIG. 5(a) shows the T1-weighted image (T1WI) of the i.p. group 12 hours after the surgery.

FIG. 5(b) shows the T1-weighted image (T1WI) of the i.c. group 12 hours after the surgery.

FIG. 6(a) shows the T2-weighted image (T2WI) of the i.p. group 12 hours after the surgery.

FIG. 6(b) shows the T2-weighted image (T2WI) of the i.c. group 12 hours after the surgery.

FIG. 7(a) shows the Diffusion-weighted image (DWI) of the i.p. group 12 hours after the surgery.

FIG. 7(b) shows the Diffusion-weighted image (DWI) of the i.c. group 12 hours after the surgery.

FIG. 8(a) shows the MRI image when the contrast agents are injected to the rat caudate nucleus.

FIG. 8(b) shows the MRI image 2 hours after the contrast agents are injected to the rat's caudate nucleus.

FIG. 9 shows change curves of the MRI signal intensity vs. time of cortex and hypothalamus of the rat brain after the contrast agents are injected.

FIG. 10(a) shows the brain slice of the rat undergoing TTC staining after the pMCAO (permanent occlusion of middle cerebral artery) surgery.

FIG. 10(b) shows the brain slice of the rat undergoing TTC staining after the drug is injected to the brain ECS.

### DETAILED DESCRIPTION OF THE EMBODIMENTS

The preferred embodiments of the present invention are described below with reference to the accompanying drawings.

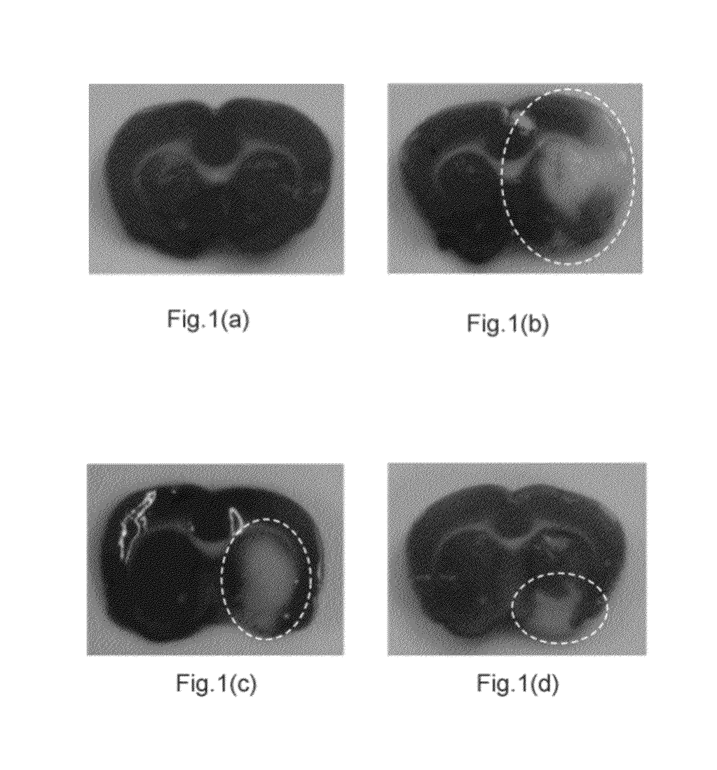

FIG. 1(a) to FIG. 1(d) show the brain slices undergoing TTC staining of rats brain after administering drugs in different way. In the experiment, rats are divided into four groups:

the first experimental group is a sham group and includes six rats;

the second experimental group is a control group and includes seven rats administered with saline;

the third experimental group is an i.p. group and includes six rats administered of citicoline with a dose of 2 g/kg by abdominal injection;

the fourth experimental group is an i.c. group and includes seven rats administered with citicoline with doses of 0.0025 g/kg via ECS at the speed of 0.30 μL/min. The volume of citicoline is 5 μL and the puncture needle retain for 5 min after delivery.

Two hours after delivery, a permanent focal cerebral ischemia model is made by thread embolism method, and MRI is performed to monitor the progress and the size of the infarction. The brains of the rats are removed 12 hours after model making The brain slices are obtained by cutting the brain in each 2 mm (5 slices each rat). Then the slices are stained at 37° C. in 0.2% TTC for evaluating the size of infarction and the effectiveness of the drug delivery method via brain ECS. The results under the TTC staining will be compared with the results under MRI.

FIG. 1(a) shows the brain slice undergoing TTC staining of the sham group 12 hours after surgery and no ischemia is found.

FIG. 1(b) shows the brain slice undergoing TTC staining of the control group 12 hours after the surgery and a large ischemic lesions involved cortex is found in the area of the dotted line as shown in the figure and the infarct volume accounts 27.7±10.5% for the brain volume.

FIG. 1(c) shows the brain slice undergoing TTC staining of the i.p. group 12 hours after the surgery. The dosage of the citicoline injected intraperitoneally is 2 g/kg. As shown in the area of the dotted line, although the infract size is smaller than that in the control group, a large ischemic lesions is found and the infarct volume accounts 24.0±10.4% for the brain volume.

FIG. 1(d) shows the brain slice undergoing TTC staining of the i.c. group 12 hours after the surgery. In this group, citicoline is injected into brain ECS in the speed of 0.3 μL/min. The consumed time is about 15 mininte. The volume of citicoline is 5 μL. The dose of citicoline is 0.0025 g/kg. The puncture needle retain for 5 min after delivery. As shown in FIG. 1(d), a small ischemic lesion is found and the infarct volume is accounts 4.1±2.0% for the brain volume, which is much smaller than that in the control group or the i.p. group.

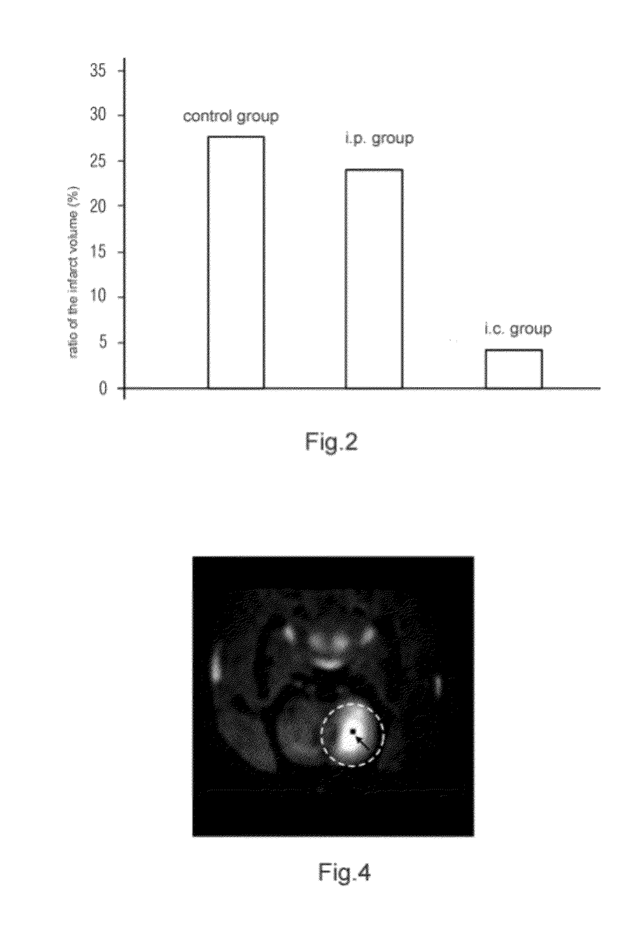

FIG. 2 shows the ratios of the infarct volume in the control group, i.p group and the i.c group 12 hours after cerebral ischemia. According to the results shown in FIG. 1, the infarct volume ratio in control group is 27.7±10.5%, the infarct volume ratio in i.p. group is 24.0±10.4%, and the infarct volume ratio in i.c. group is 4.1±2.0%. There are significant differences among the three groups. Basing on the one-way analysis of variance (ANOVA) followed by individual comparisons of means (Dunnet's method when the data were not normally distributed). It is concluded that there is a statistically significant difference between the i.p. group and the i.c. group (P<0.01).

The result shows that brain extracellular space (ECS) exists in the brain interstitial tissue and the narrow and interconnected space between cell membrane is filled with interstitial fluid. The brain ECS is recognized as the exchange channel for essential substances between brain cells and microvessels. It has been found that the fraction volume of the ECS on the brain(about 20%) is higher than that of microvessels (only 3). Because the ECS has the large contact area with brain tissue and can avoid the blocking of BBB, the delivery via brain ECS can enhance the drug intake and reduce the drug dose and the side effects of the drug.

After injecting into brain ECS, the drug will diffuse to target area through interstitial fluid and cure the diseases. In the exemplary embodiments of the method for drug delivery via brain ECS, the injected drugs is citicoline (including citicoline, Cytidine-5-diphosphate choline and CDP-choline), which is an essential substance to sustain life activities. The citicoline can be obtained from food and transformed in the brain. The citicoline has small molecular weight (510.31 Dalton), but strong polarity. It is difficult for citicoline to pass the blood-brain barrier. The intake of delivered citicoline by oral or intravenous method in brain tissue is very low, and the treatment result is not ideal. As shown in FIG. 2, the treatment effect of citicoline has been improved by the method of the present invention. As can be understood, if other drugs have the appropriate character with ECS like concentration, molecular weight, space conformation and polarity and will not injure the brain tissue on the structure and the function, these drugs also can be used in the present invention.

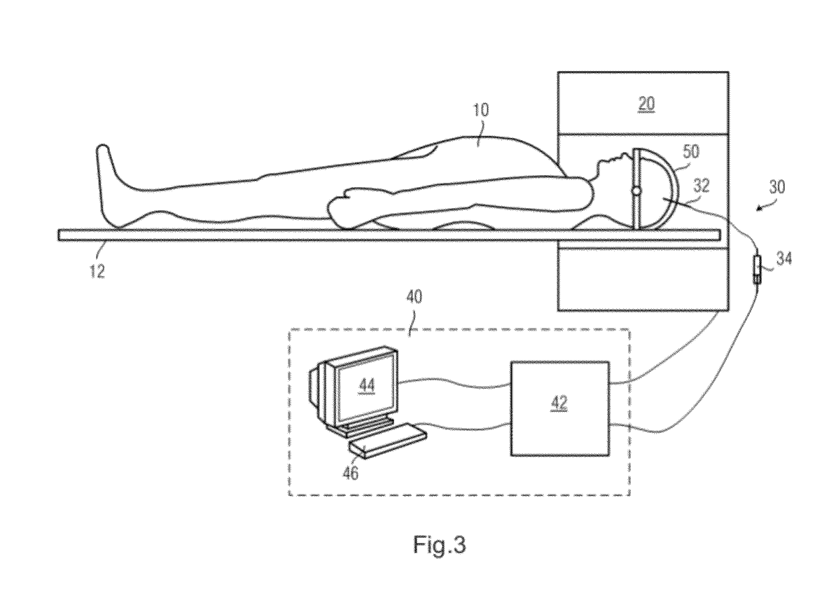

FIG. 3 schematically illustrates a drug delivery device via brain extracellular space. As shown in FIG. 3, a patient 10 who has brain disease is put into an imaging apparatus 20 along with a check bed 12. The imaging apparatus is CT, MRI or other imaging apparatus. The imaging apparatus 20 is connected with a controlling apparatus 40.

The controlling apparatus 40 includes a control unit 42, a monitor unit 44 and an input unit 46. To observe the patient's condition in the imaging apparatus 20 at any time, the images in the imaging apparatus 20 (such as MRI) are displayed on the monitor unit 44. This process is controlled by the control unit 42.

As shown in FIG. 3, the drug delivery apparatus 30 may include a puncture needle 32 and an injection pump 34. Persons skill in art can understand that other methods also can deliver drugs into ECS such as nasal instillation, inhalation delivery and so on. When the other delivery methods are used, the imaging apparatus 20 can also help the operator to observe the location and the corresponding concentration of the drug.

In the drug delivery via puncturing, the brain of the patient 10 can be set in a stereotaxic apparatus 50. The stereotaxic apparatus 50 can orientate the different areas in the patient's brain and assist the delivery apparatus 30 to inject the drug into correct position. The stereotaxic apparatus can use commercially available products which neither interact with the imaging apparatus 20 nor affect the quality of the imaging of the patient's brain, for example, the stereotaxic apparatus can be made of brass or other anti-magnetic materials.

The puncture needle 32 is fixed in the stereotaxic apparatus 50. The operator can determine the correct position of puncture on the stereotaxic apparatus 50 basing on the images of the patient's brain on the monitor unit 44. Thus, the tip of the puncture needle 32 can be localized accurately in the brain ECS of the patient 10 via the stereotaxic apparatus 50 and the monitor unit 44. The puncture needle 32 can be any commercially available product which neither interact with the imaging apparatus 20, nor reduce the imaging quality, nor injure the cells of the patient's brain. For instance, the puncture needle 32 can be made of the stainless steel approved by the Chinese State Standard GB/T 3280-2007 (approve No. 022Cr17Ni12Mo2Ti) and the United State ASTM Standard (stainless steel 316L).

The distal end of the puncture needle 32 is connected with the injection pump 34. The injection pump 34 is jointed with the control apparatus 40, which is configured to control the injection rate and total dose of drugs in the injection pump. In a result, the rate and dosage of the drug delivery into ECS of the patient by the puncture needle 32 can be exactly performed.

In the drug delivery device of the prevent invention, the control apparatus 40 is connected with the injection pump 34 and the imaging apparatus 20. The images of the patient's brain obtained via the imaging apparatus 20 can display on the monitor unit 44 for the operator to observe. The control unit 42 can store the data of drug delivery beforehand or the operator can input the data via the input unit 46. The data of drug delivery includes the injection rate and dosage of the drug delivered by the injection pump and so on. Besides, the real-time state of the patient's brain after drug delivery can be observed by the operator via the monitor unit 44. Therefore, the dynamic supervision and medical care can be realized during the process of drug delivery and the best therapeutic effect can be reached.

FIG. 4 shows the MRI image of the punctured rat's brain being. The arrow in the circle indicates the area the puncture needle reaches.

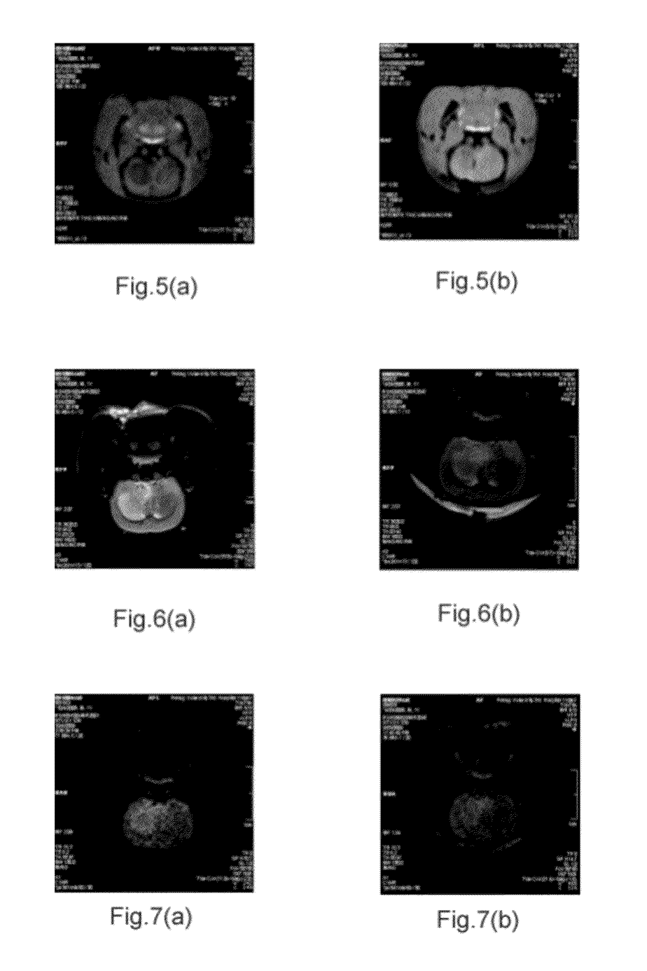

FIG. 5(a) shows the T1-weighted image (T1WI) of the i.p. group 12 hours after the surgery. As shown in FIG. 5(a), the areas of thalamus and most caudate nucleus show low signal intensity in the T1 weighted images of the i.p. group. This indicates that the cerebral infarction occurred in these areas. FIG. 5(b) shows the T1-weighted image of the i.c. group 12 hours after the surgery. As shown in FIG. 5(b), only the area of thalamus shows low signal intensity in the T1 weighted images of the i.c. group, which indicates that cerebral infarction occurred only in the thalamus.

FIG. 6(a) shows the T2-weighted image (T2WI) of the i.p. group 12 hours after the surgery. As shown in FIG. 6(a), the area of thalamus and most caudate nucleus show low signal intensity in the T2 weighted images of the i.p. group which indicates that the cerebral infarction occurred in these areas. FIG. 6(b) shows the T2-weighted image (T2WI) of the i.c. group 12 hours after the surgery. As shown in FIG. 6(b), only the area of thalamus shows low signal intensity in the T2 weighted images of the i.c. group, which indicates that cerebral infarction, occurred only in the thalamus.

FIG. 7(a) shows the diffusion-weighted image (DWI) of the i.p. group 12 hours after the surgery. As shown in FIG. 7(a), the area of thalamus and most caudate nucleus shows hyperintensity in the DWI of the i.p. group which indicates that the cerebral infarction occurred in these areas. FIG. 6(b) shows the diffusion-weighted image of the i.c. group 12 hours after the surgery. As shown in FIG. 7(b), only the area of thalamus shows hyperintensity in the DWI of the i.c. group, which indicates that cerebral infarction occurred only in the thalamus.

The above MR images reach the same conclusion with the TTC staining brain slices shown in FIG. 1.

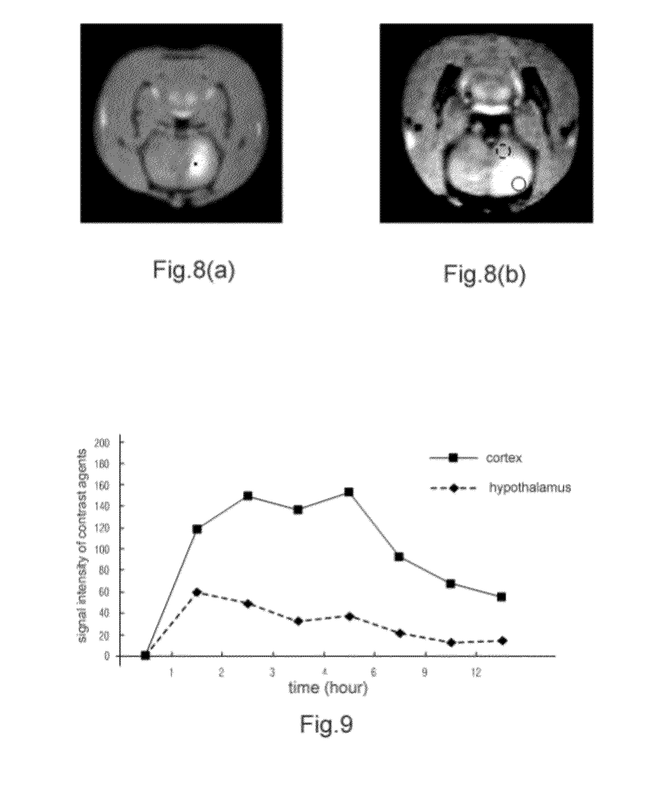

In order to illustrate the diffusion process of injected drugs via ECS in the present invention, the contrast agents are injected into the rats' brain. FIG. 8(a) shows the MR image when the contrast agents are injected into the rat's caudate nucleus. The black point in the figure shows the injection site of the contrast agents Gd-DTPA.

FIG. 8(b) shows the MR image 2 hours after the contrast agents injected into the rat's caudate nucleus. As shown in FIG. 8(b), in the thalamus area outlined by the dashed line the signal intensity is not strengthen, which indicates that the concentration of the contrast agents fail to reach the level in this area. However, the signal intensity of MR image of the cortex outlined by the solid line is increased significantly, which indicates the concentration of the contrast agents maintains at a certain level in this area.

FIG. 9 shows a change curve of the MRI signal intensity vs. time of cortex and hypothalamus of the rat brain after the contrast agents are injected. Similar to the result in FIG. 8(b), the signal intensity of the thalamus area outlined by dashed line is increased slightly 1 hour after the injection, and then decreased. In the exemplary embodiment, the MR image intensity is increased slightly 2 hours after the injection and the signal increment is about 50. But the MR signal intensity of the cortex area outlined by the solid line is increased immediately after the injection and maintained at a relatively high level for a long time. Two hours after the injection of the contrast agents, the increment of the signal intensity in the cortex area is about 140, which is 3 times of that in the thalamus area. It is implied that the concentration of the drug in the cortex area is about 3 times of that in the thalamus area.

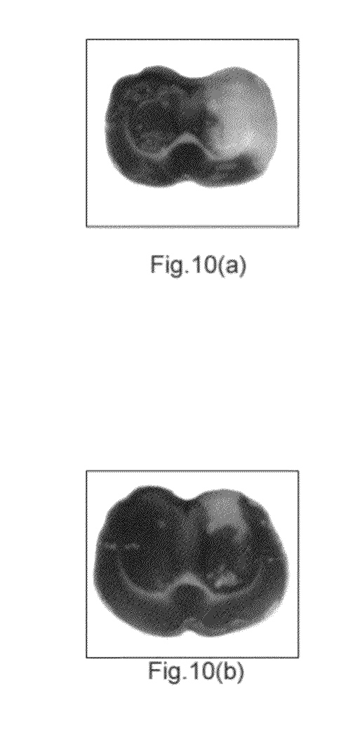

FIG. 10(a) shows the rat's brain slice undergoing TTC staining after the pMCAO surgery. The infracted focus shows white color of the TTC staining As shown in FIG. 10(a), the thalamus and most of the cortex are infracted.

FIG. 10(b) shows the rat brain slice undergoing TTC staining after the drugs injected into the brain ECS. The neuroprotective drug citicoline is infused into the ECS in the center of the right caudate nucleus of rat brain. The cerebral ischemia is induced by the middle cerebral artery occlusion 2 hours after the infusion. It is demonstrated by the TTC staining that the infarction does not occur in the cortex. Similar with the diffusion process of the contrast agents shown in FIG. 9, the drug infused into ECS 2 hours before the pMCAO diffused to the cortex (shown as the dashed line in FIG. 9), and the concentration of the drug maintains a level high enough to protect the brain tissue from the ischemic injury. However, the concentration of the drug in the thalamus is decreased quickly after a prompt increase (shown as the dashed line in FIG. 9). Thus, without the neuroprotection, the infarction occur in this region.

In the present invention, drugs can be delivered exactly into brain ECS of the patient by dynamic monitoring using imaging apparatus, such as MRI, etc. Not only the process of drug delivery via the brain ECS of the patient, but also the self diffusion of the drugs can be monitored to evaluate the effectiveness of the drugs.

1. A method comprising:
responsive to receiving, from an imaging apparatus, dynamic images of a brain of a patient, delivering a drug to a brain extracellular space of the patient, the delivery adapted to cause the drug to move to a therapeutic target of the patient by self-diffusion along a concentration gradient.

2. The method according to claim 1, wherein the drug is delivered to the brain extracellular space via brain puncturing.

3. The method according to claim 1, wherein the drug is CDPC.

4. The method according to claim 1, wherein the imaging apparatus is the magnetic resonance imager.

5. A device comprising:
an imaging apparatus (20) configured to get dynamic images of a patient;
a drug delivery apparatus (30) configured to deliver a drug to a brain extracellular space of the patient;
a controlling apparatus (40) that is connected with the imaging apparatus (20) and the drug delivery apparatus (30), said controlling apparatus (40) comprising:
a control unit (42) configured to receive imaging signals from the imaging apparatus (20) and control a speed and a total dose of the drug by communicatively connecting with the drug delivery apparatus (30);
a monitor unit (44) that is communicatively connected with the control unit (42) and configured to display images from the imaging apparatus (20).

6. The device according to claim 5, wherein the drug delivery apparatus (30) comprises:
a puncture needle (32) configured to import the drug to the brain extracellular space of the patient; and
a dosing pump (34), that is connected with the puncture needle (32) and configured to deliver the drug to the puncture needle (34).

7. The device according to claim 6, wherein, the device further includes a stereotaxic apparatus (50) to fix a position of the patient's head in the imaging apparatus(20).

8. The device according to claim 7, wherein, the stereotaxic apparatus (50) is made of brass.

9. The device according to claim 5, wherein the controlling apparatus (40) further comprises an input unit (46) that is connected to the control unit (42) and configured to input a controlling signal to the imaging apparatus (20) and the drug delivery apparatus (30).
